(19) United States
(12) Patent Application Publication
(10) Pub. No.: US 2023/0223123 A1
(43) Pub. Date: Jul. 13, 2023

(54) System And Method For Diagnostic Coding

(71) Applicant: Bruce Wayne FallHowe, Colorado Springs, CO (US)

(72) Inventor: Bruce Wayne FallHowe, Colorado Springs, CO (US)

(21) Appl. No.: 18/123,724

(22) Filed: Mar. 20, 2023

Related U.S. Application Data
(63) Continuation-in-part of application No. 17/477,155, filed on Sep. 16, 2021, now abandoned.
(60) Provisional application No. 63/084,278, filed on Sep. 28, 2020.

(51) Int. Cl. G16H 15/00

(52) U.S. Cl. CPC G16H 15/00

(57) ABSTRACT

A computer implemented coding system for mandating correct medical diagnostic coding by a provider, comprises program code executable to receive patient encounter medical data associated with a patient. Medical elements performed during the patient encounter are compared and matched with up-to-date guidelines that include medical guideline, derived from a stored database of diagnostic code requirements or via a machine learning/artificial intelligence acquisition of up-to-date guidelines, by which a diagnosis is made and published for consideration by a treating provider. The matched guideline is published on a display screen visible to the treating provider where it is accepted or refused in favor of the treating providers alternative diagnosis. If accepted, and insurance code is determined and submitted for payment. If not accepted, a list of missing medical procedures associated with the provider's diagnosis is determined and ordered before an insurance code may be assigned.

### REFERENCE TO RELATED APPLICATIONS

This application is related to U.S. patent Ser. No. 17/477,155 filed Sep. 16, 2021 titled System and Method for Diagnostic Coding, its which claims the priority of provisional patent application U.S. Ser. No. 63/084,278 filed Sep. 28, 2020, titled “A checklist of elements for medical billing coding that audits what must be documented in the chart” and which is incorporated in its entirety herein by reference.

### BACKGROUND OF THE INVENTION

This invention relates generally to medical billing systems and, more particularly, to a medical diagnostic coding system and method that mandates correct claims coding of a medical diagnosis and provides instant feedback to and correction by a treating provider.

Medical claim and billing systems are inherently complicated, multi-layered, redundant, and inefficient. In a typical system, a treating provider makes a preliminary diagnosis which is then matched with a billing code, such as by a “coder.” The provider's diagnosis and code may then be reviewed by a clinic or hospital coding group and, if the code is found to be incorrectly selected, the “clinic or hospital coder” may change the code. The treating provider is typically never informed of the mistake and is likely to continue the incorrect coding practice. The clinic or hospital coder may then submit a claim to a respective insurance company where the diagnosis and code are once again reviewed—this time by an “insurance coder department which is composed of coders, nurses and physician.” If the code is still incorrect, the claim will be denied. The substitute diagnosis is given by the insurance company and the diagnosis/diagnoses are paid. Once the clinic or hospital are made aware of this, their claims department writes an appeal to substantiate the original diagnostic code(s). The clinic or hospital appeals department documents criteria that they believe will allow the original diagnostic code(s) to be reimbursed.

Various medical billing systems have been proposed throughout the years. Although presumably effective for their intended purposes, a common characteristic of such systems is that they still include multiple layers of claim audits, coding changes, and a lack of feedback to the treating provider.

Therefore, it would be desirable to have system and method that mandates correct diagnostic coding during a patient's first experience with a treating provider so as to minimize or even eliminate the multiple layers of claim audits, coding changes, and reimbursement modifications.

### SUMMARY OF THE INVENTION

A computer implemented coding system according to the present invention for mandating correct medical coding by a treating provider, comprises a processor for executing program code and a non-transitory computer-readable storage medium containing program code executable to perform the steps of receiving medical symptom data, examination findings, and medical diagnostic data associated with the patient so that the coding system can make a preliminary diagnosis of a medical condition of the patient. This enables a diagnosis to be determined based solely on the diagnostic elements performed during the patient encounter when matched with a matching record in a diagnostic code requirement database, said comparing and matching being performed in some embodiments using a machine learning and artificial intelligence engine trained by up-to-date diagnostic guidelines promulgated by a national authority, learned articles, payor denial letter, or the like.

Then, according to the present invention, the treating provider is asked to either accept the medical diagnosis based solely on the diagnostic elements that were generated by the initial patient encounter and which matches the guidelines found in the diagnostic code database or via operation of a machine learning/artificial intelligence engine diagnostic guidelines assimilated from UpToDate, Society Guidelines, and National Library of Medicine. If accepted, a code associated with the accepted diagnosis may be chosen and submitted to a payor with confidence of its correctness, and appropriate services ordered for the patient. If the diagnosis is not accepted, by contrast, the treating provider is invited to submit a physician suspected diagnosis (PSD) which is then matched to an appropriate diagnostic guideline in the manner described above and such that a list of “missing diagnostic elements” is determined and which are required by the PSD. Again, the treating provider is asked to either accept the originally determined diagnosis or else the missing diagnostic elements are automatically ordered and the inventive method is essentially restarted.

Therefore, a general object of this invention is to provide a system for diagnostic medical coding that results in determining a diagnosis and associated billing code based solely on the initial patient encounter and without human interaction, said patient encounter including initial diagnostic elements that are matched with an up-to-date diagnostic guideline without needing a clinic, hospital or insurance audit procedure.

Another object of this invention is to provide a diagnostic medical coding system, as aforesaid, that matches initial diagnostic procedures and results with an up-to-date guideline and associated illness having identical diagnostic elements, thereby eliminating the risk of over-diagnosis, under-diagnosis or misdiagnosis.

Still another object of this invention is to provide a diagnostic medical coding system, as aforesaid, that seeks the approval or acceptance of the treating provider only after a diagnosis has been determined based solely on the diagnostic elements initially ordered and the results.

Yet another object of this invention is to provide a diagnostic medical coding system, as aforesaid, that provides immediate feedback to a treating provider regarding missing diagnostic elements that must be ordered should the treating provider insists on making an independent diagnosis beyond that which is justified by the diagnostic elements performed initially.

Other objects and advantages of the present invention will become apparent from the following description taken in connection with the accompanying drawings, wherein is set forth by way of illustration and example, embodiments of this invention.

### BRIEF DESCRIPTION OF THE DRAWINGS

FIG. 1 is a block diagram illustrating a computer-implemented diagnostic coding system according to the preferred embodiment of the present invention connected to key player databases via the Internet;

FIG. 2 is a block diagram illustrating a coding database, a component of the diagnostic coding system as in FIG. 1;

FIG. 3 is a block diagram illustrating the diagnostic coding system according to the present invention in use installed on an electronic device having a processor and memory; and

FIGS. 5 to 7a is a flowchart of a process illustrating the diagnostic coding system according to the present invention.

### DESCRIPTION OF THE PREFERRED EMBODIMENT

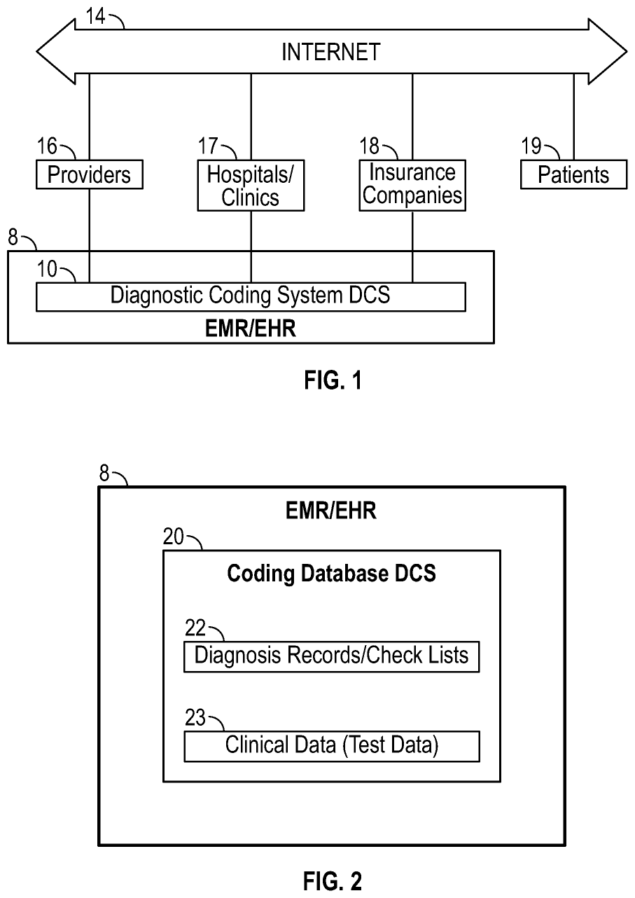

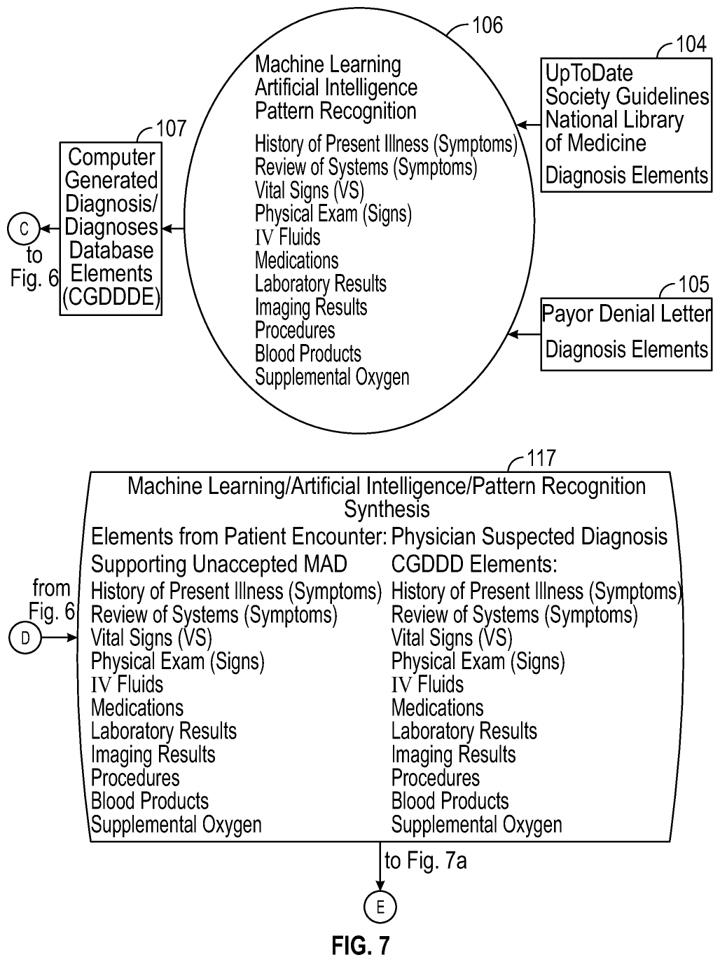

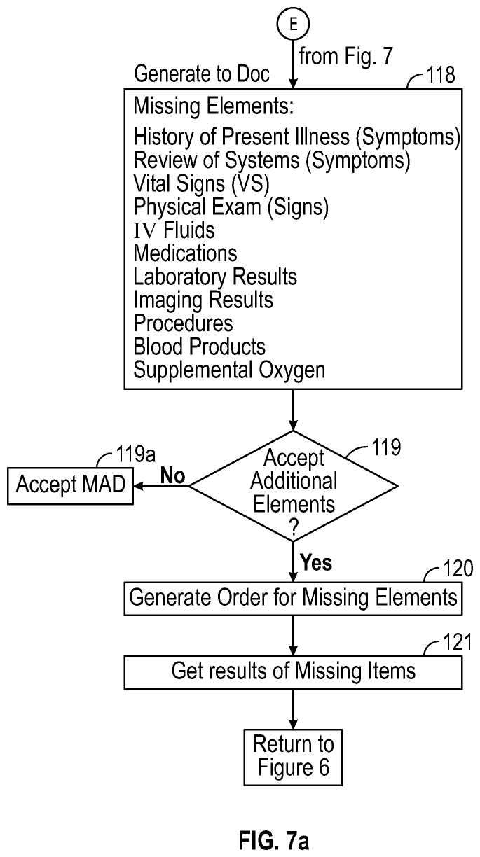

A system and method for diagnostic coding according to a preferred embodiment of the present invention will now be described with reference to FIGS. 1 to 7a of the accompanying drawings. The system 10 that mandates correct diagnostic coding includes computer software and a diagnostic coding database 20 connected to the interne and including coding executable by a processor 12.

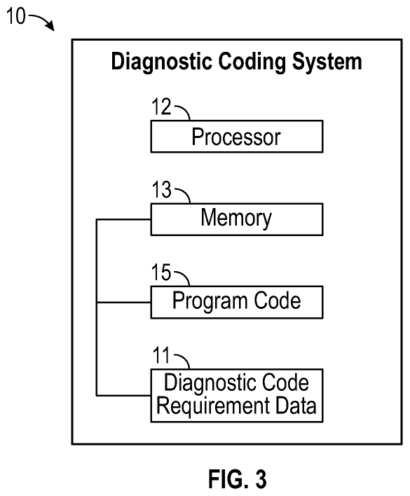

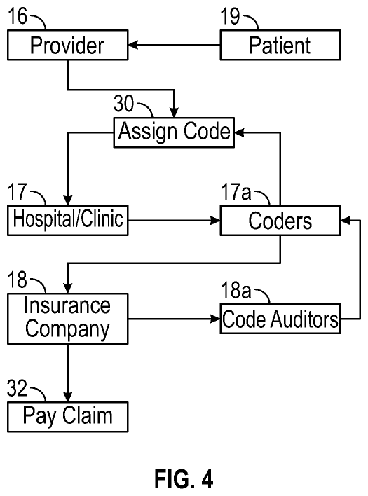

The system for diagnostic coding 10 may be stored on a non-transitory computer readable storage medium, i.e., memory 13, for execution by a processor 12 of an electronic device connectable to a wide area network 14 such as the Internet or local area network such as that of a clinic or hospital. Preferably, the diagnostic coding system 10 is stored on an electronic device carried by a treating provider, such as a laptop computer, a tablet, or smart phone and that is connected to the network. The diagnostic coding system 10 may include program code 15 (e.g., computer instructions) and a database that includes diagnostic code requirement data 11 (FIG. 3). As will be discussed in greater detail later, the diagnostic code requirement data 11 may include a plurality of numeric diagnostic codes each being associated with a detailed outline of medical tests and medical procedures that must be administered or satisfied in order for a respective diagnostic code to be selected. In an embodiment, the diagnostic code requirement data 11, also known as a diagnostic element database, may receive repeated updates from authoritative sources such as the UpToDate, Society Guidelines, National Library of Medicine (NLM) and the National Institutes of Health (NIH) such that the diagnostic code database 11 is essentially a biomedical library that may be compared to respective diagnostic procedures and results of a patient encounter, whereby to determine a correct diagnosis of a patient's illness based solely on those initial diagnostic procedures and results, as will be described in greater detail later. As will also be described, this process of repeatedly updating the diagnostic element database 11 may include actual and specific algorithms that are “trained” using machine learning, artificial intelligence, and pattern recognition technologies in a machine learning engine environment.

As shown particularly in FIGS. 1 and 2, it will be understood that the system for diagnostic coding 10 or at least the program code 15 may be included in the larger computer architecture of an Electronic Medical Record 8 (EMR), also known as an Electronic Health Record (EHR). An EMR is a digital version of the paper charts in the clinician's office. EMRs contain the medical and treatment history of the patients in one practice. As shown, the present system for diagnostic coding 10 may be a subset, program subroutine, or specialty program such as may be selected from within a patient's EMR 8.

Each installation and operation of the diagnostic coding system 10 may include a communications module in data communication with a wide area network such as the Internet 14 or with a local computer network such as within a clinic, hospital or hospital group. This preferred connectivity enables the diagnostic coding system 10 to be used by the treating provider 16, clinic, hospitals 17 or hospital coding groups 17a, insurance companies 18 or insurance company coding groups 18a, as well as directly with a consumer/patient 19 who may be in communication with the treating provider. In addition, this network connection enables digital communication with a source of diagnostic codes and elements and required diagnostic elements associated with each code, such as is provided by UpToDate, Society Guidelines, and the National Library of Medicine. Another network connection may be made with a payor rejection letter and its elements as will be explained later.

With specific reference to FIG. 2, the diagnostic coding system 10 includes a coding database 20 having a plurality of diagnosis records 22 each of which is associated with an alphanumeric code and with a comprehensive checklist or outline of symptoms, past medical history, physical examination findings, medical tests and medical procedures that must be completed or satisfied in order for the respective code to be used to describe a respective patient's diagnosis. It is understood that the coding database 20 may be stored in a non-transitory storage medium, i.e., a memory device of the electronic device upon which the diagnostic coding system 10 is installed. Program code 15 may also be stored in the memory device 13 and configured for execution by a processor 12 so as to carry out the present invention. Further, actual clinical test data 23 (relative to the hypothetical patient who presents himself for treatment) may also be stored in appropriate data structures of the memory device. As will be explained later, the diagnostic records associated with a claim code or diagnostic code may be updated in association with using an artificial intelligence engine.

FIG. 4 illustrates the inefficiencies of a traditional diagnostic coding scheme and that which is typical of the prior art. With regard to a traditional diagnostic and billing system shown in FIG. 4, a treating provider 16 interacts with a medical patient 19, such as in person, via telemedicine, or the like. Based solely on an initial examination, a patient's report, or whatever preliminary tests may be run in real time, the treating provider 16 may make a preliminary diagnosis of a medical condition and may choose a diagnosis code accordingly. But before the selected diagnostic code can be submitted as an insurance claim, the provider's preliminary diagnosis may be submitted to a clinic/hospital 17 and, more particularly, to office or hospital coding group 17a where the preliminary diagnosis is considered along with any auxiliary medical test data that may have come into the patient's record after the preliminary diagnosis was made. The clinic or hospital coding group 17a may determine, based on a totality of the medical record, that the initial billing code is now incorrect. Similarly, the clinic or hospital coding group 17a may determine that the diagnosis is not clear and request or substitute another diagnosis code. In either instance, the clinic or hospital coders 17a may send the matter back to the treating provider 16 for reevaluation and recoding.

Once the clinic or hospital coding group 17a is satisfied with the diagnostic code, a claim may be submitted to the patient's insurance company 18 and, more particularly, to the insurance company coding auditors 18a. Once again, there is the potential that the insurance company coding auditors 18a may disagree that the claim diagnosis billed for is not supported by the documentation in the medical record. In such case, the insurance company coding department 18a will substitute another diagnosis and pay the hospital or clinic based on the substituted diagnosis.

As a real-world example of a patient encounter in a traditional billing system (FIG. 4), a patient may present to a provider regarding painful urination. The provider, then, may make an initial diagnosis of a “urinary tract infection” and the associated billing code is assigned. Unfortunately, however, this code may be premature until a plurality of additional testing has been completed. Currently, later lab data may reveal or even contradict this coding error which must then be changed after one or more coding audits by entities other than the treating provider, e.g., by clinic, hospital, or insurance company coding audits. In other words, a code assigned to “urinary tract infection” may require a different set of indications, lab tests, other tests, etc. that, if they had been performed, may have resulted in a different diagnosis and, therefore, a more appropriate coding. By contrast, this preliminary error is prevented by the present invention as discussed below.

By contrast, the efficiencies of the present invention will be summarized before being described in detail and then illustrated by way of the same example used with the inefficient traditional system described above. More particularly, a patient is thoroughly examined by a provider so as to hear and document the complaints of a patient, i.e., the initial patient encounter or patient complaint. This examination may prompt the provider to run an initial series of tests. In a critical aspect of the present invention, the software of the preferred embodiment of the diagnostic coding system 10, will correctly determine a preliminary diagnosis—independent of the treating provider. More particularly, the diagnostic coding system 10 is configured (such as by program code and using the diagnostic code requirement database 11) to generate a Computer-Generated Diagnostic/Diagnosis Database Elements (CGDDDE). Stated another way, the present invention, i.e., the computer assisted coding system 10, is configured to determine the only diagnosis possible based on the preliminary tests and procedures shown to have been run at that point in time. In other words, the medical provider, e.g., the doctor, is simply not allowed to over diagnose or incorrectly diagnose the patient's malady and an incorrect diagnosis is not allowed to be submitted. Needless to say, the patient is not in danger of being incorrectly treated, such as with medicines or procedures. As will be described later, the determination of the CGDDDE may be made with use of a machine learning and artificial intelligence engine that is trained by repeated communications with diagnostic guidelines produced by an official source, such as UpToDate, Society Guidelines, and the National Library of Medicine which provides diagnosis elements that must be undertaken before a code may be adopted, respectively. In fact, the machine learning/artificial intelligence engine may be trained by receiving repeated infusions or downloads regarding biomedical research, denial letters, and information concerning a correct diagnosis based on the diagnostic elements that have been performed. It is understood that the numerous diagnostic elements associated with a chosen diagnosis evolve and change over time, making the search engine all the more important to generate the correct preliminary diagnosis.

In another critical aspect, the computer-generated diagnosis is electronically presented to the medical provider and the medical provider is asked whether he accepts the computer-generated diagnosis (which will be referred to as the Machine Learning Artificial Intelligence Diagnosis/Diagnoses (MAD)). Several post-patient-visit procedures take place and the proper code is submitted for payment. By contrast, if the MAD is not accepted, then the ML/AI may be accessed again for the purpose of generating a list of diagnostic elements associated with the Physician Suspected diagnosis/diagnoses (PSD) which are displayed to the physician again, these additional elements are presented to the medical provider who is given the choice to accept the computer-generated MAD or to press on with his own diagnosis. If accepted, the process proceeds to the post-patient-visit procedures; otherwise, an order is generated for the missing diagnostic elements, i.e., an order for additional lab tests/procedures, etc., and the process essentially starts over.

This methodology has the advantages of providing knowledge to the treating provider regarding the complete outline of tests, procedures, etc., required for a given diagnosis and also enables the diagnosis to be changed based on the results of the required listing of tests. This process provides the instant feedback that a provider may need to avoid making a repeated error in diagnostic coding. Further, this process assures that the final billing code selected by the treating provider is correct—making future coding audits unnecessary. Accordingly, the final diagnostic code may be submitted to a clinic or hospital billing department then submitted as a claim to the insurance coding group for payment of the claim with total assurance that an audit is not necessary and that there is no need to return the coding issue back up the line.

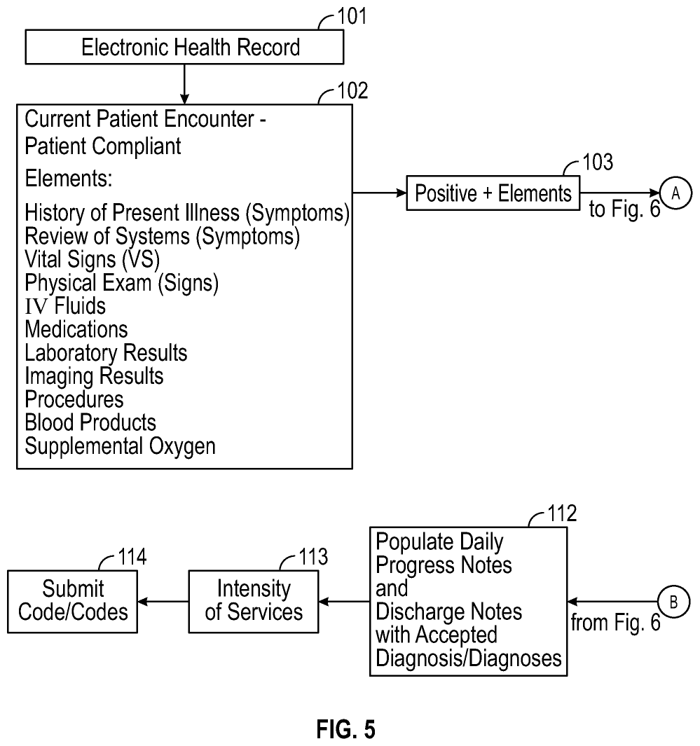

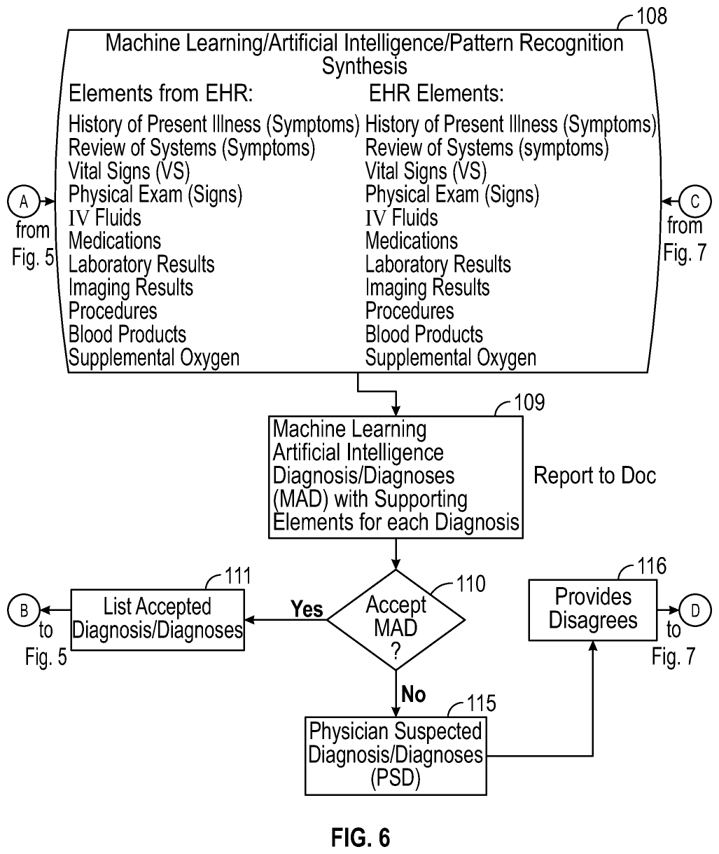

With the understanding explained above, this process and method will now be described in detail with reference to FIGS. 5 to 7a and with corresponding reference numerals.

A process 100 illustrating the logic of the diagnostic coding system 10 is shown in FIG. 5 et seq. It is understood that the process 100 is preferably implemented as computer software having a plurality of program instructions (aka programming) stored in a non-transitory memory 13 and executable by a processor 12 of an electronic device used or carried by a treating provider and that is connected to the Internet 14. According to the process 100 and as indicated at block 101, a patient may present himself to a treating provider and presumably brings his personal electronic health record (EMR) with him (which may be in the form of electronic records in the computer system of the provider, on a flash drive belonging to the patient, or otherwise electronically archived) so that it is available to the treating provider and may, if appropriate, be supplemented during or after the client encounter. As shown at block 102, a patient encounter occurs when a patient presents his symptoms to the treating provider, such as in a typical office visit, or hospital setting, a telemedicine encounter over the Internet 14, or the like. It is understood that a complete patient encounter, which may also be referred to as a “patient complaint”, may include recording a history of the present illness (i.e., a patient discussing his symptoms), a review of a patient's bodily systems, the taking of the patient's vital signs, and a physical examination, a review of the patient's IV fluids (or an order is made directing that IV fluids be taken), a review of the patient's current medications, the review of any imaging results (such as x-ray, CT scan, MM scan, or the like), laboratory results, a review of any medical procedures undertaken, a review of any blood products given, and any supplemental oxygen. The process 100 proceeds to step 103.

At step 103, the exact nature and results of the diagnostic elements currently in the patient's encounter are determined—no more and no less. The diagnostic elements by which the initial diagnosis may be determined have been completed by way of the initial patient encounter. The process 100 proceeds to step 108.

At step 108, the processor 12 determines what diagnosis is indicated by the diagnostic elements resulting from the patient encounter determined in step 103. Naturally, step 108 involves comparing the diagnostic elements (and their results) with the predetermined diagnostic elements prescribed by a standardized medical authority, such as UpToDate, Society Guidelines promulgated by the National Library of Medicine (NLM) and the National Institutes of Health (NIH). Note that the comparison of diagnostic elements from the patient encounter versus a Computer-Generated Diagnosis/diagnoses Database Elements (CGDDDE) is shown in block 108. Block 108 is intentionally sandwiched between block 103 and block 107 in that the comparison of these two sets of elements occurs in block 108. And, the CGDDDE diagnostic elements may be generated by the actions shown in blocks 104, 105, and 106. More particularly, block 106 represents a Machine Learning/Artificial Intelligence/Pattern Recognition engine that is uniquely configured to generate a more complete and accurate set of guidelines to be used in step 107 and then step 108. More particularly, the ML/AI/PR engine is trained by its repeated network access to the latest diagnostic elements, biomedical research, biomedical articles, and the like proffered by UpToDate, Society Guidelines National Library of Medicine (step 104). Further, the ML/AI/PR engine may be further trained by accessing hundreds or even thousands of the payor denial letters which also includes current diagnostic elements and which clarify proper and improper diagnoses with relation to required medical procedures and results (step 105). Accordingly, the ML/AI/PR engine generates the latest and most accurate diagnostic element outlines and associated illness data. In addition, and as shown at step 106, the ML/AI/PR engine receives and is trained by the latest expert information regarding the history of the present illness indicated by the patient's symptoms and any elements determined so far.

Further, when the processor 12 compares the computer-generated diagnostic elements to the patient encounter diagnostic elements in step 108, the processor 12 will determine at step 109 what is referred to as a MAD (ML/AI Diagnoses/diagnoses), also known as the computer-generated preliminary diagnosis based solely on the patient encounter synthesized in comparison with the diagnostic elements that have been implicated so far—nothing more and nothing less. Preferably, each element of the CGDDDE will be graphically or textually matched with each corresponding diagnostic element that was performed during the patient encounter so as to more powerfully justify the correctness of the preliminary diagnosis (a.k.a. the MAD diagnosis was correct). Such a comparison may utilize pattern recognition and other algorithms that are characteristic of the ML/AI/PR engine. The process 100 proceeds to step 110.

At step 110, the processor 12 determines if the treating provider will agree to accept the MAD discussed above (such as by posing this question in a text will prompt on-screen) and, if so, the process 100 proceeds to steps 111, 112, 113 and 114 at which the accepted diagnosis may be saved or published, post-encounter services may be prompted/ordered, and the accepted diagnostic/insurance code may be submitted for payment, respectively. (FIGS. 5-7a). But, if the MAD is not accepted by the treating provider at step 110, the process 100 proceeds to step 115 and 116.

At step 115, the treating provider may input his suspected diagnosis (also referred to as a Physician Suspected Diagnosis (PSD)) in the process 100 proceeds to step 117. At step 117, the ML/AI/PR engine is configured to synthesize or make a comparison of the diagnostic elements generated by the patient encounter (as described previously) with the diagnostic elements associated with the PSD so as to generate a set that will be referred to and saved as “Missing Elements” at step 118. In other words, the processor 12 is configured to determine, using the AI engine (which, again, may be in the data communication with UpToDate, society guidelines and the National Library of Medicine) a list of diagnostic elements (i.e., test procedures, etc.) that are uniquely required for the PSD but that have not been accomplished per the patient encounter and CGDDDE. After saving and publishing this list of Missing Elements at step 118, the process 100 proceeds to step 119.

At step 119, the processor 12, in a routine similar to that described above in step 110, queries the treating provider to determine if the treating provider agrees to proceed in order the Missing Elements (i.e., agrees to order the missing lab tests or medical procedures, etc. that are prescribed for the PSD) and, if not, the process 100 proceeds to step 119a and the MAD (original CGDDDE) is accepted/adopted. But, if the treating provider agrees that the Missing Elements that need to be considered before the PSD can be confirmed, then the process proceeds to step 120 whereby orders for the additional elements are generated in the process proceeds to step 121 where the procedures according to the Missing Elements take place in what will be referred to as a secondary or auxiliary or supplemental patient encounter. Accordingly, the data from the auxiliary or supplemental patient encounter may update the EMR and the process returns to step 108 where a new MAD may be determined at step 109 and presented for acceptance at step 110 as described above.

To be thorough in its disclosure, the concept of machine learning and artificial intelligence will be described in greater detail.

In the last decade, the applications of machine learning (ML) algorithms have proliferated in many areas of scientific, industrial, and medical applications. In general, modern artificial intelligence (AI) systems are capable to classify with high precision very complex data. The algorithms have inspired the development of novel artificial neural network (ANN or NN) forming the basis of the field of artificial intelligence (AI). The developments have been in response to the vast amount of data, known as Big Data, which require new approaches to their processing, storage, and information extraction. Not without some limitations, AI continues to find new applications and provide fast information extraction based on algorithm embedded knowledge acquired with the use of available data in order to process newly available data: the process also known as NN training.

More particularly, the preliminary and auxiliary medical test data may be scanned and received into a natural language (NL) reader. In general, natural language processing (NLP) refers to using machine learning algorithms in relation to text and speech. More particularly, NLP may be used to create machine translations, document summaries, question answering, predictive typing, and the like. In the present case, medical documents summarizing lab work, medical advice, and other documentation may be scanned in or digitally transferred to a natural language file to be viewed by the treating provider or compared to the diagnostic code database 20 whereby the processor 12, executing programming code 15, is able to determine if a selected diagnostic code associated with a respective record and associated diagnosis corresponds with scanned medical test data and diagnosis (initial or final).

In use, the system and method for correct medical diagnostic coding 10 is configured to determine a correct initial medical diagnosis that is based solely on an initial patient encounter and the initial diagnostic elements that were performed during that initial patient encounter. More particularly, the treating provider is not given opportunity to over-diagnose, under-diagnose, or mis-diagnose; rather, the processor 12 in conjunction with the program code 15, diagnostic code requirement database 11 and, in some embodiments, the ML/AI/PR engine are configured to determine the proper initial diagnosis of the patient's malady according only to the diagnostic elements having already been carried out. In fact, this preliminary diagnosis, based only on the data of record i.e., the MAD), will be published and presented to the treating provider along with an inquiry to determine if the provider agrees with the MAD. If accepted, a few post-procedure actions will occur and the inventive method is terminated. Otherwise, the provider may enter his own suspected diagnosis, to which the processor 12 is configured to determine a list of diagnostic elements required for said provider diagnosis and to publish or display this list of Missing Elements to the physician along with an inquiry if the treating provider will either accept the original MAD or, rather, agrees to order the additional or auxiliary diagnostic elements (e.g., Additional lab tests, scans, or other medical procedures). If the additional diagnostic elements are ordered, the results will be added to the patient encounter and the method of determining a diagnosis starts over. Importantly, the scenario of an initial diagnosis being determined by the system software and the results being presented to the treating provider has the unique advantage of providing immediate educational feedback to the treating provider and displaying the only possible diagnosis based on current medical elements currently of record may lead to supplemental and additional diagnostic elements to be ordered and considered before agreeing to a diagnosis and, therefore, an insurance code submitted for payment of a claim.

According to the present invention, a patient diagnosis can be made quickly and accurately without undertaking one or multiple coding audits.

It is understood that while certain forms of this invention have been illustrated and described, it is not limited thereto except insofar as such limitations are included in the following claims and allowable functional equivalents thereof.

1. A computer implemented system for mandating correct medical coding by a treating provider of a patient, said system comprising:
a non-transitory computer-readable storage medium containing program code and data structures;
a diagnostic coding database in said storage medium having a plurality of records each including at least a medical diagnosis and a corresponding outline of predetermined diagnostic elements that must be ordered and determined before a code associated with said medical diagnosis can be assigned;
a processor in data communication with said computer-readable storage medium and operative to execute said program code to perform the steps of:
receiving medical symptom data from the patient;
receiving diagnostic test data associated with the patient;
comparing said received medical symptom data and said received diagnostic test data to said plurality of records of said diagnostic coding database until a mutually exclusively matching record is located;
publishing said matching record and the medical diagnosis and corresponding outline of predetermined diagnostic elements associated therewith;
determining if the treating provider agrees that the medical diagnosis associated with said matching record should be made final and, if so, recommending predetermined patient support information and, if not, receiving physician suspected diagnosis (PSD) data;
determining from said diagnostic coding database a respective record corresponding to said PSD data, said respective record including a PSD outline of diagnostic elements;
determining a list of missing diagnostic elements yet to be ordered or performed by comparing said PSD outline of diagnostic elements with said corresponding outline associated with said matching record; and
determining if the treating provider agrees that the medical diagnosis associated with said matching record should be made final and, if so, recommending predetermined patient support information and, if not, generating orders to perform said list of missing diagnostic elements.

2. The system as in claim 2, further comprising an artificial intelligence engine in data communication with said diagnostic coding database and with an official source of diagnostic element guidelines such that said artificial intelligence engine is trained by repeatedly downloading diagnostic element updates so as to enhance speed and accuracy of locating said matching record.

3. The system as in claim 2, wherein said artificial intelligence engine is configured to repeatedly download learned articles and biomedical research data regarding respective illnesses included in said diagnostic coding database such that said artificial intelligence engine is trained by repeatedly downloading said learned articles and said biomedical research data so as to enhance speed and accuracy of locating said matching record.

4. The system as in claim 3, wherein said processor is configured to execute said program code to perform the steps of:
if the treating provider accepts that the medical diagnosis associated with said matching record should be made final, using the diagnostic coding database to determine an insurance code associated with said accepted diagnosis associated with said matching record.

5. The system as in claim 4, wherein said processor is configured to execute said program code to perform the step of displaying on a display screen said determined insurance code associated with said accepted diagnosis.

6. The system as in claim 5, wherein said processor is configured to execute said program code to perform the step of communicating said determined insurance code associated with said accepted diagnosis to a third-party coder via a wide area network.

7. The system as in claim 6, wherein said third party coder is an insurance company.

8. The system as in claim 6, wherein said third party coder is a clinic or hospital.

9. A method for mandating correct medical coding by a treating provider of a patient comprises the steps of:
receiving up-to-date diagnostic guidelines each diagnostic guideline including at least a medical diagnosis and a corresponding outline of predetermined diagnostic elements that must be ordered and determined before a code associated with the medical diagnosis can be assigned;
receiving medical symptom data from the patient;
receiving medical test data associated with the patient;
comparing said received medical symptom data and said received diagnostic test data to said received diagnostic guidelines until a mutually exclusively matching guideline is located;
publishing to an electronic device said matching guideline and the medical diagnosis and the outline of predetermined diagnostic elements associated therewith;
determining if the treating provider accepts that the medical diagnosis associated with said matching guideline should be made final and, if so, recommending predetermined patient support information and, if not, receiving physician suspected diagnosis (PSD) data;
determining from said received diagnostic guidelines a respective guideline corresponding to said PSD data, said respective guideline including a PSD outline of diagnostic elements;
determining a list of missing diagnostic elements yet to be ordered or performed by comparing said PSD outline of diagnostic elements with said corresponding outline associated with said matching guideline;
determining if the treating provider accepts that the medical diagnosis associated with said matching guideline should be made final and, if so, recommending predetermined patient support information and, if not, generating orders to perform said list of missing diagnostic elements.

10. The method as in claim 9, wherein said step of comparing said received medical symptom data and said received diagnostic test data to said received diagnostic guidelines includes providing a neural network (NN) in data communication with a diagnostic coding database and having algorithms configured for machine learning (ML) and artificial intelligence (AI) operable to determine said matching guideline.

11. The method as in claim 10, wherein said ML/AI algorithms are trained using repeated downloads of guidelines promulgated by National Library of Medicine.

12. The method as in claim 11, wherein said ML/AI algorithms are trained using repeated downloads of biomedical research regarding respective illnesses included in said diagnostic coding database so as to enhance accuracy of locating said matching record.

13. The method as in claim 111, wherein said ML/AI algorithms are trained using repeated downloads of payor denial letters that each explain what elements are required for a selected diagnosis.

14. The method as in claim 12, further comprising using a diagnostic coding database to determine an insurance code associated with said accepted diagnosis.

15. The method as in claim 14 further comprising displaying on a display screen said accepted diagnosis and the insurance code associated with said accepted diagnosis.

16. The method as in claim 14, further comprising communicating said insurance code associated with said accepted diagnosis to a third-party coder via a wide area network.

17. The method as in claim 16, wherein said third-party coder is an insurance company.

18. The method as in claim 16, wherein said third party coder is a clinic or a hospital.
